(19) United States
(12) Patent Application Publication
(10) Pub. No.: US 2014/0302527 A1
(43) Pub. Date: Oct. 9, 2014

(54) TARGET-SPECIFIC PROBE COMPRISING FERRITIN PROTEIN AND DETECTION FOR BIOMARKER USING THE SAME

(71) Applicant: KOREA INSTITUTE OF SCIENCE AND TECHNOLOGY, Seoul (KR)

(72) Inventors: Kwan-Hyi LEE, Goyang-si (KR); Jong-Hoon Choi, Seoul (KR); Mintai Peter Hwang, Seoul (KR); Yu-Chan Kim, Goyang-si (KR); Jong-Wook Lee, Anyang-si (KR); Hyun-Kwang Seok, Seoul (KR)

(73) Assignee: KOREA INSTITUTE OF SCIENCE AND TECHNOLOGY, Seoul (KR)

(21) Appl. No.: 13/899,830

(22) Filed: May 22, 2013

(30) Foreign Application Priority Data
Apr. 8, 2013 (KR) 10-2013-0038147

(51) Int. Cl. G01N 33/68; C07K 16/28; C07K 14/315; C07K 14/47

(52) U.S. Cl. CPC G01N 33/6854; C07K 14/47; C07K 16/28; C07K 14/315
USPC 435/7.21; 530/400; 530/387.3; 530/395

(57) ABSTRACT

This invention relates to a target-specific probe containing a ferritin fusion protein and a targeting agent, a target-specific imaging probe containing a labeling agent coupled to the target-specific probe, and a detection method or detection kit of a biomarker using these probes.

### CROSS REFERENCE TO RELATED APPLICATION

This application claims the benefit of Korean Patent Application No. 10-2013-0038147, filed on Apr. 8, 2013, the contents of which is incorporated herein by reference.

### FIELD OF THE INVENTION

This invention relates to a target-specific probe comprising a ferritin protein and a targeting agent, and a detection method, a quantification method and a detection kit of a biomarker using the probe.

### BACKGROUND OF THE INVENTION

A biomarker is a type of biomaterials present in biological or medical specimens, which functions as a marker capable of diagnosing the condition of a disease by detecting a change in the structure or concentration thereof qualitatively and/or quantitatively and determining the treatment effects of a medicine and correlation with other diseases comprehensively. For the early diagnosis of diseases, it is essential to analyze a biomarker occurring in the beginning stage of the diseases quantitatively. However, technologies currently available for monitoring the diseases do not properly meet technical needs for the early diagnosis of diseases because of limits such as sensitivity, quarantine speed, and costs.

ELISA (Enzyme-Linked ImmunoSorbent Assay), western blotting, and a mass spectrometry-based method have been generally used to quantitatively analyze biomarkers. In case of ELISA, accurate detection is difficult because a reaction may be inhibited by polysaccharides or phenol compounds present in the test samples or the concentration of bacteriophages present in tissues is low. While the mass spectrometry-based method has very good sensitivity so that they are applicable to analyze a slight amount of a biomarker, it has difficulty in securing reproducibility because this is usually analyzed by being linked to chromatography method and it also has a huge deviation of analysis data due to machine errors. In addition, these methods require excessive labor and a large amount of time.

Recently, according to the rapid development of nanotechnologies, detection technologies which were impossible under the previous detection methods are emerging. For example, Lieber et. al from Harvard University published a nano-detecting sensor for detecting a single bacteriophage (Science, vol. 329, pp. 830-4, Aug. 13 2010), and Mirkin et. al from Northwestern University established Nanosphere company, on the basis of a molecule detection technology using a nanoprobe (Sensors, vol. 12, pp. 1657-1687, Feb. 7 2012).

However, while the methods based on nanoparticles have excellent detection sensitivity, two-dimensional detection methods such as nano elements have poor accessibility toward a subject to be detected to quantitatively analyze minute amounts (FIG. 1).

A quantum dot is an inorganic semiconductive substance having a nano size, which is recently applied to various medical engineering fields because of its excellent optical properties including high quantum efficiency, excellent resistance to photo fading, the control of fluorescence property by size, and non-overlapped fluorescence spectrum. Attempts to use quantum dots have been made for the quantification of important disease markers (Analytical Chemistry, vol. 76, pp. 4806-4810, Aug. 15 2004; Analytical Chemistry, vol. 82, pp. 5591-5597, Jul. 1 2010).

Furthermore, in order to overcome quenching phenomenon where the fluorescence intensity of quantum dots becomes dramatically weak, attempts to measure the number of quantum dots in a different manner were published. For example, the change of electrical conductivity due to cadmium ions which constitute quantum dots was measured by dissolving the quantum dots in a strong acid when Prostate-specific Antigen (PSA) was separated, detected, and quantified (Small, vol. 4, pp. 82-86, January 2008), but there was an issue that a large amount of cadmium ions which are toxic substances were generated. In another analysis example using quantum dots, with the purpose of measuring intrinsic fluorescence by the separation of quantum dots, organic solvents and alkali solutions having a high concentration were used to cleave Streptavidin-Biotin bond (Analyst, vol. 135, pp. 381-389, 2010.), but there were issues that the aggregation of quantum dots might occur due to the organic solvents, various buffering solutions were required because of the deterioration of the stability and optical properties of the quantum dots under experiment conditions, and it had poor reproducibility.

Therefore, in order to detect biomarkers with high sensitivity, a new detection system of biomarkers capable of overcoming the limits of the previous detection systems including nanoparticles and nano elements, being simply analyzed, being easily handled, and providing accurate detection results with high sensitivity is needed.

### SUMMARY OF THE INVENTION

It is an object of the invention to provide a target-specific probe comprising a ferritin protein and a targeting agent.

It is another object of the invention to provide a detection method, a quantification method and a detection kit of a biomarker using the target-specific probe, capable of overcoming the limits of the previous detection systems including nanoparticles and nano elements, being simply analyzed, being easily handled, and providing accurate detection results with high sensitivity.

In order to achieve the above-mentioned objects, there is provided in one embodiment of the present invention a target-specific probe comprising a ferritin complex comprising a ferritin protein and a coupling partner coupled to the ferritin protein, and a targeting agent coupled to the ferritin complex.

Preferably, the ferritin complex may comprise a peptide or protein, or a compound as a coupling partner, and when the coupling partner is a peptide or protein, the target-specific probe comprises the ferritin protein; and the peptide or protein which is connected to the C-terminal or N-terminal of the ferritin protein and is a fusion partner not inhibiting ferritin structure formation. When the coupling partner is a compound, the ferritin and the compound are coupled to form a ferritin complex, which is then coupled to a targeting agent to form a target-specific probe.

Another embodiment of the invention relates to a method for detecting a biomarker comprising contacting a target-specific probe comprising a targeting agent specific to the biomarker and a detectable labeling agent to a sample containing the target biomarker to be detected, and detecting the labeling agent of the biomarker-targeted probe.

In the method for detecting a biomarker, when the labeling agent is detected, the biomarker can be determined to be present in the sample, or the amount of the biomarker in the sample can be measured by producing a standard curve of the labeling agent and comparing the detectable amount of the biomarker-targeted labeling agent with the standard curve.

Further embodiment of the present invention relates to a detection kit comprising a target-specific probe and a detector for detecting a labeling agent of the targeted probe.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram in which two-dimensional detection methods such as nano elements according to the prior arts show the limits in detecting a slight amount with super sensitivity, due to the limits of accessibility to the subject to be detected.

FIG. 2 is a gene map of the fusion protein of ferritin and Protein G according to one embodiment of the invention.

FIG. 3 is a schematic diagram showing a process of producing a probe for super sensitivity detection by fixing an antibody on the surface of ferritin and then coupling it with a number of quantum dots according to one embodiment of the invention.

FIG. 4 is a schematic conceptual diagram showing a method for targeting a biomarker at the surface of a cell/tissue using the imaging probe prepared in FIG. 3 according to one embodiment of the invention

FIG. 5 is graphs showing a change in the size of the imaging probes according to the functionalization thereof by one embodiment of the invention, which was measured with DLS. FIG. 5(a) indicates the size of ferritin, (b) indicates the size of the assembly of ferritin and antibody, and (c) indicates the size of an imaging probe obtained by coupling the assembly of ferritin and antibody with a number of hydrophilic quantum dots. FIG. 5(d) is a TEM image of the imaging probe.

FIG. 6 is fluorescence images observed after a biomarker at the surface of cell/tissue is targeted using a probe according to one embodiment of the invention. FIG. 6(a) is a fluorescence image when a probe where one fluorescent dye is coupled to one antibody is used, and (b) is a fluorescence image when a probe where one quantum dot is coupled to one antibody is used. FIG. 6(c) is a fluorescence image when a probe for super sensitivity detection where a number of quantum dots are coupled to one antibody is used.

FIG. 7 is a quantum dot concentration-fluorescence intensity related standard curve obtained by analyzing various concentrations of quantum dots and resultant fluorescence intensities according to one embodiment of the invention.

FIG. 8 is a photograph showing an imaging kit for super sensitivity quantity analysis of a biomarker developed in the invention, where A is a ferritin solution, B is a water-dissolved quantum solution, and C is a reaction vessel.

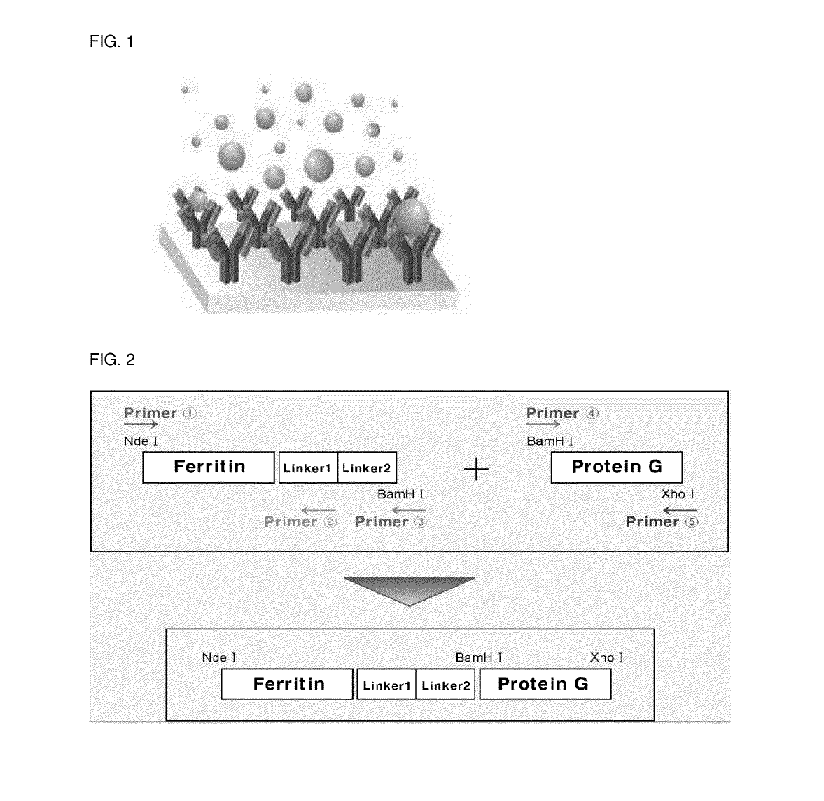

### DETAILED DESCRIPTION OF THE INVENTION

Hereafter, the present invention will be described in more detail.

The probe according to the invention is a target-specific probe comprising a ferritin complex of a ferritin protein-a coupling partner; and a targeting agent coupled to the ferritin complex.

In this specification, the targeting agent is a targeting agent having a targeting ability specific to a biomarker which is a subject to be detected or measured and for example, it may be an antibody, aptamer, aptide, or peptide. The antibody may be a monoclonal antibody or polyclonal antibody, an immunologically active fragment (e.g., Fab or (Fab)₂ fragment), an antibody heavy chain, an antibody light chain, a gene-engineered single chain molecule, a chimeric antibody, or a humanized antibody. The coupling of the ferritin fusion protein with the targeting agent enables the target-specific probe to move toward the biomarker target to be detected.

In the specification, the ferritin protein refers to comprise the ferritin protein itself, a heavy chain of ferritin, a light chain of ferritin, an analogue thereof, and apoferritin. Ferritin is a protein aggregate widely present in extracellular matrix, which consists of 24 single subunits and forms a cage-like nanostructure of which the outer diameter is 12 nm and the inner diameter is 8 nm. The ferritin cage has an internal empty space having the size of about 8 nm, contains in its inside about 4500 Fe atoms in their ferric oxide state, and serves to supply such Fe atoms during metabolic process.

The ferritin complex may be either in the form of a fusion protein produced by the coupling of the ferritin protein and a peptide or protein, or in the coupled form of the ferritin protein and a compound. As the coupling partners applicable to the present invention, any desired substances including Protein G, Fc receptor, Protein A, Protein A/G, Biotin, Avidin, and Streptavidin can be used.

In an aspect of the invention, when the ferritin complex is a fusion protein produced by bonding the ferritin protein and a peptide or protein, the ferritin complex may be a ferritin fusion complex comprising the ferritin protein; and the protein or peptide which is connected to the C-terminal or N-terminal of the ferritin protein and is a fusion partner not inhibiting ferritin structure formation.

The fusion partner and the ferritin protein may be directly connected, or fused using a linker peptide consisting of 30 or less amino acids, preferably, 15 to 25 amino acids. The coupling partner of the ferritin fusion protein aids the ferritin complex in functioning to be targeted at the biomarker by being coupled with the targeting agent. The coupling partner capable of being expressed as the ferritin fusion protein may include Protein G, Fc receptor, Protein A, or Protein A/G, but not be limited thereto.

When the targeting agent is an antibody, the targeting ability can be maximized by using the property that the coupling partner specifically binds to the F_{c} portion of the antibody targeting agent so that the F_{ab} portion of the biomarker-targeted antibody can be always activated. In chemical binding methods using N-hydroxysuccinimide (NHS), and ethyl(dimethyl aminopropyl) carbodiimide (EDC) (Nat Protoc 2007, 2 (5), 1152-1165), there is a high possibility that the F_{c} portion of an antibody may not be activated due to a non-specific binding between the antibody and nanoparticles, eventually decreasing the targeting efficiency. Hence, in order to overcome such a low efficiency, an expensive antibody needs to be used for reaction in excessive amounts.

The target-specific probe according to an aspect of the invention may further comprise a detectable labeling agent which is coupled to the ferritin complex. The present invention relates to a target-specific imaging probe comprising one or two more detectable labeling agents which are coupled to a linker for coupling the labeling agents of the target-specific probe. The target-specific imaging probe may further comprise a linker for coupling the detectable labeling agent, selected from HIS tag, CYS tag, GST tag, Biotin tag, Avidin tag, and Streptavidin tag connected to the terminal of the ferritin protein or the coupling partner. Preferably, the labeling agent may be coupled to the ferritin complex via the linker, and the subject biomarker-targeted probe may be detected using the labeling agent.

The labeling agent applicable to the invention may include a quantum dot, magnetic bead nanoparticle, gold nanoparticle, fluorescent dye, fluorescent protein, nano phosphor, or silicon nanoparticle, and the labeling agent may be detected by fluorescence microscopy, SEM, TEM, CT, MRI, etc.

The labeling agent in itself may be coupled to a linker for coupling the labeling agent, selected from HIS tag, CYS tag, GST tag, Biotin tag, Avidin tag, and Streptavidin tag, or it may be coupled to the linker after the chemical treatment to the labeling agent to increase a binding ability toward the linker.

As a method for coupling the labeling agent to the ferritin complex, for example, a polymerase chain reaction (PCR) may be utilized. A desired labeling agent can be synthesized by performing PCR, using a fusion protein as a template and including the labeling agent in primers. Alternately, it can be performed by using a fusion protein as a template, inserting two desired restriction enzyme sites into the fusion protein, likewise inserting the same kinds of restriction enzyme sites into the both ends of the labeling agent, cleaving the genes with the restriction enzymes, and then fusing the cleaved genes using ligation.

When the ferritin complex is a compound coupled to ferritin, the labeling agent binds to a correspondent compound capable of binding to the compound, thereby forming a bond between the compound of the ferritin complex and the correspondent compound of the labeling agent to prepare a target-specific probe containing the detectable labeling agent. In particular, the ferritin complex may be a complex formed by coupling Biotin tag, Avidin tag, or Streptavidin tag compound as the coupling partner to the ferritin protein, and the invention may be a target-specific probe comprising a compound coupled to the ferritin complex, and capable of chemically binding to the compound coupling partner as a detectable labeling agent. For example, when Biotin is the coupling partner, Streptavidin can be used as the labeling agent.

While the detectable labeling agent may be coupled in 1 to 24 moles per mole of the ferritin complex, 2 to 15 moles are preferable in consideration of the high detection rate and sensitivity of the labeling agent, and space between the labeling agents.

The labeling agent may be a quantum dot, magnetic bead nanoparticle, gold nanoparticle, fluorescent dye, fluorescent protein, nano phosphor, or silicon nanoparticle.

When the labeling agent is a quantum dot, it may be used in itself, or it may be a quantum dot comprising a hydrophilic surface layer thereon obtained by treating the surface with an amphiphilic substance containing both of hydrophilic group and hydrophobic group. Particularly, it is preferable that the quantum dots show the least aggregation phenomenon by possessing hydrophilicity and it is more preferable that they are functionalized with nickel. For example, the hydrophilic surface may be obtained by treating it with one or more substances selected from the group consisting of 1-Myristoyl-2-Hydroxy-sn-Glycero-3-Phosphocholine (MHPC), 1,2-Distearoyl-sn-Glycero-3-Phosphoethanolamine-en-Methoxypolyethyleneglycol-2000 (DPPE-PEG2000), and 1,2-Dioleoyl-sn-Glycero-3-en-{5-amino-1-carboxylpentyl}iminodiacetic acid succinyl nickel salt (Ni-NTA). The amphiphilic substances may be treated alone or in a combination of two or more.

In particular, MHPC can be densely coated onto the surface of a sphere since it is a single acyl group chain lipid, DPPE-PEG2000 can confer stability on the quantum dots coated with the lipids, and Ni-NTA can bind to histidine on ferritin to bond hydrophilic quantum dots and ferritin. Thus, a functionalized probe capable of quantifying a biomarker with super sensitivity can be prepared by adding quantum dots having hydrophilic surface to the biomarker-targeted antibody and ferritin complex,

According to a specific embodiment of the invention, MHPC, DPPE-PEG2000, and Ni-NTA may be used alone or preferably, they can be used in a mixture form comprising the three components. For example, 50 to 95 mole % of MHPC, 4 to 35 mole % of DPPE-PEG2000, and 1 to 15 mole % of Ni-NTA are mixed with quantum dots to become an emulsion state, which is then sonicated to form a hydrophilic surface layer on the surface of the quantum dots. The above composition is merely mentioned as one illustration, and the number and the ratio of the lipids to be used may be adjusted to the desired purpose.

A preferred target-specific probe according to the invention may be formed by the sequential binding of protein G or Fc receptor, and ferritin from its N-terminal to C-terminal, wherein the Fc portion of the targeting antibody agent may be connected to protein G or Fc receptor of the ferritin fusion protein and more preferably, it may comprise HIS tag, CYS tag, GST tag, Biotin tag, Avidin tag, or Streptavidin tag which is connected to the terminal of the ferritin protein or the terminal of the coupling partner.

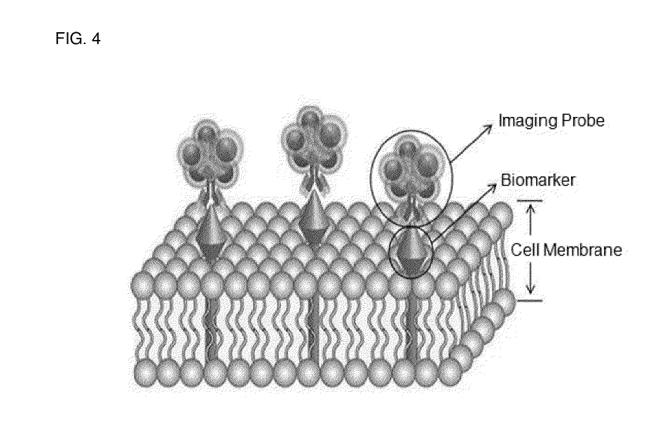

FIG. 4 is a schematic conceptual diagram showing a method for targeting a biomarker at the surface of a cell/tissue using the probe according to one embodiment of the invention. After the imaging probe is targeted at the biomarker on the surface of a cell/tissue, the concentration of the biomarker can be quantified through the fluorescence intensity value of quantum dots.

Another aspect of the invention relates to a method for detecting a biomarker comprising contacting the target-specific probe containing a targeting agent specific to the biomarker and a labeling agent to a sample containing the biomarker, and detecting the labeling agent of the biomarker-targeted probe.

Also, another aspect of the invention relates to a detection kit of a biomarker comprising a target-specific probe and a detector for detecting a labeling agent of the targeted probe. The target-specific probe has been described in detail in the above.

The method for detecting biomarker may further comprise a step in which the biomarker is determined to be present in the sample when the labeling agent is detected. Furthermore, it may be a method in which a standard curve is produced with detectable amounts measured at various concentrations of the labeling agent, and the amount of the biomarker in the sample is measured by comparing the detectable amount of the biomarker-targeted labeling agent with the standard curve.

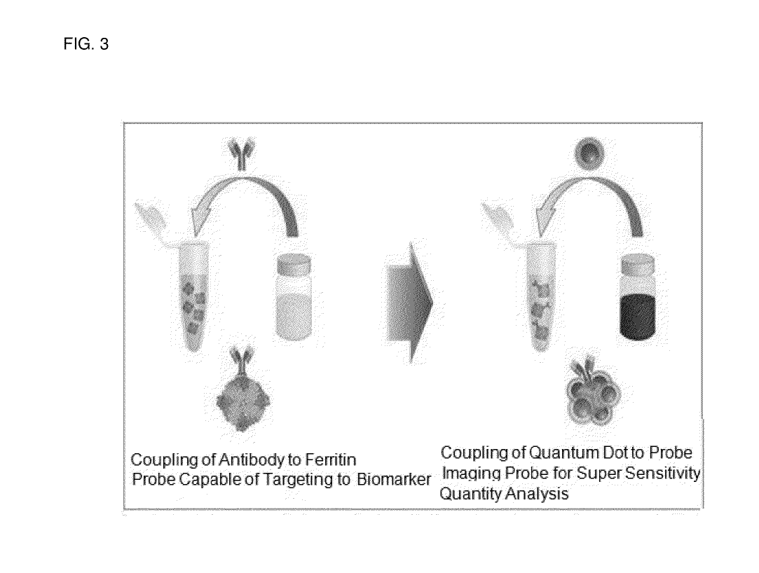

As a specific example of the invention, FIG. 3 illustrates a schematic diagram of a process of producing a probe for super sensitivity detection by fixing an antibody on the surface of ferritin and then coupling a number of quantum dots thereto. Protein G, a coupling partner of the ferritin fusion protein, which bonds an antibody for detecting a biomarker and quantum dots can specifically bind to the Fc portion of the antibody, thereby enabling the Fab portion of the antibody for collecting the biomarker to be always in its active state. Also, in order to form a complex consisting of biomarker detection antibody-ferritin-quantum dot, histidine of ferritin coupled to the Fc portion of the biomarker detection antibody and Ni-NTA present at the hydrophilic surface of quantum dot can be coupled. FIG. 4 is a schematic conceptual diagram showing a method for targeting a biomarker to the surface of a cell or tissue using the probe. After the imaging probe is targeted at the biomarker on the surface of a cell/tissue, the concentration of the biomarker can be quantified through the fluorescence intensity value of quantum dots.

According to the invention, super sensitivity quantity analysis is carried out by measuring the fluorescence intensity values of quantum dots after targeting a biomarker at the surface of a cell/tissue using ferritin and quantum dots, and a detection kit may be manufactured using this. Accordingly, the biomarker detection kit comprises a target-specific probe and a detector for detecting a targeted probe and labeling agent.

The present invention provides a target-specific probe containing a ferritin fusion protein and a targeting agent, as a new detection system of a biomarker capable of being simply analyzed, being easily handled, and providing accurate detection results with high sensitivity, and a detection method and detection kit of a biomarker using the probe, and it can be usefully utilized for early diagnosis of diseases with known biomarkers.

Hereafter, the invention will be described in more detail through examples and comparative examples. However, the following examples are to merely illustrate the present invention, and the scope of the invention is not limited by them in any ways.

### Example 1

### Preparation of Probe for Detecting Biomarker

1-1: Fusion Protein of Protein G and Ferritin Protein

In order to connect the genes for the ferritin protein and Protein G purchased from Promega Co., PCR was carried out using a total of three pairs of primers consisting of five primers. The primers were all purchased from Cosmogenetech (Seoul, Korea). Also, restriction enzymes Nde I, BamH I, and Xho I were purchased from New England Biolabs (Ipswich, Mass., USA).

TABLE 1

| | | SEQ ID |
|---|---|---|
| Designation | Sequence 5′→ 3′ | NO: |
| Forward Primer 1 | CATATGACGACCGCGTCCACCTCG | 1 |
| Backward Primer 2 | ACTGCCACCTCCAGTACCGCCTC | 2 |
| | CGCTTTCATTATCACTGTC | |
| Forward Primer 1 | CATATGACGACCGCGTCCACCTCG | 1 |
| Backward Primer 3 | GGATCCTCCACCGCTTCCACCGCC | 3 |
| | TGTTCCACCGCCACTGCCACCTCCAG | |
| | TACC | |
| Forward Primer 4 | GGATCCACTTACAAATTAATCCTT | 4 |
| Backward Primer 5 | CTCGAGATTAGTGATGGTGATGG | 5 |
| | TGATGTTCAGTTACCGTAAAGGT | |

Ferritin and Protein G were connected using a linker of 54 bp, and the linker was divided into two to carry out PCR. First, the ferritin portion was connected to Nde I and linker 1 using primer \textcircled{1} (SEQ ID NO:1) and primer \textcircled{2} (SEQ ID NO:2).

Next, primer \textcircled{1} (SEQ ID NO:1) and primer \textcircled{3} (SEQ ID NO:3) were used to connect the PCR product (Nde I+Ferritin+Linker 1) and linker 2+BamH I,

The Protein G portion was connected to BamH I and Xho I, using primer \textcircled{4} (SEQ ID NO:4) and primer \textcircled{5} (SEQ ID NO:5), and PCR conditions were the same as above. PCR was carried out under the following conditions, and each PCR product was identified by performing Agarose gel electrophoresis of the PCR products.

TABLE 2

| segment | Number of Cycles | Temperature | Time |
|---|---|---|---|
| 1 | 30 | 95° C. | 4 min, 30 sec |
| 2 | 30 | 55° C. | 30 sec |
| 3 | 30 | 72° C. | 40 sec |
| 4 | 1 | 72° C. | 7 min |
| 5 | 1 | 4° C. | ∞ |

The terminal portions of the genes of the ferritin portion and the Protein G portion produced through PCR were each cleaved using restriction enzyme BamH I. The two genes cleaved with sticky ends were connected using a ligation enzyme as shown in FIG. 2 to fuse ferritin and Protein G.

1-2: Coupling of Targeting Antibody

20 μl of 0.1 μM ferritin solution containing Protein G and fused ferritin prepared in 1) above and 3 μl of 0.1 mg/ml CLDN4 (claudin-4) anti-human antibody were mixed in a reaction vessel and then reacted at a room temperature for one hour to bond Protein G of the ferritin subunit and the antibody, thereby obtaining a probe capable of targeting a biomarker. The CLDN4 anti-human antibody was purchased from Invitrogen.

1-3: Coupling of Labeling Agent

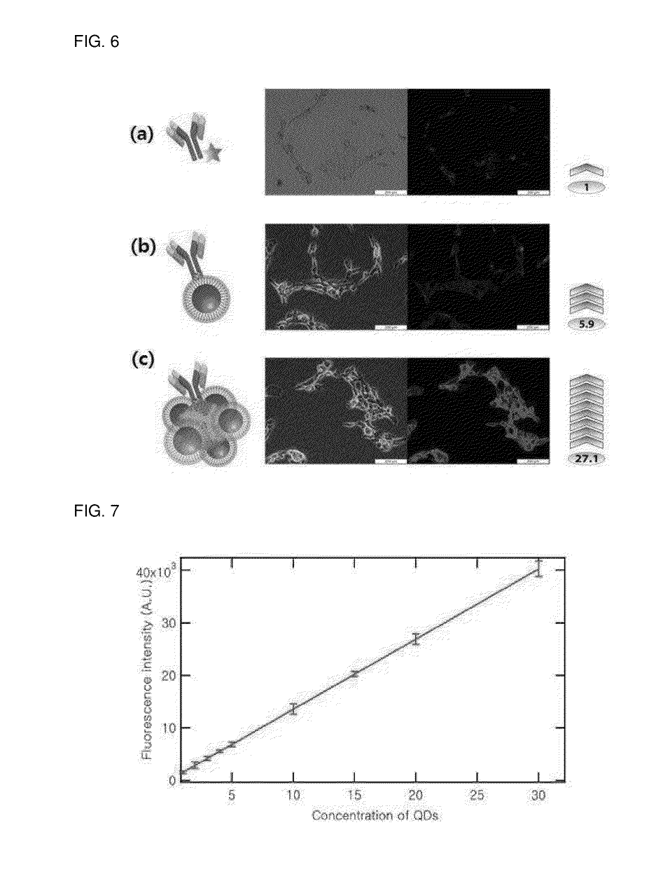

100 μl of 0.2 μM quantum dots were added to the reaction solution obtained by the coupling of the ferritin fusion protein and the targeting antibody to mix them in the molar ratio of 1:10 and then reacted at a room temperature for one hour to produce an imaging probe for super sensitivity quantity analysis where one antibody was coupled to a number of quantum dots as shown in FIG. 6 (c). Also, for comparison purpose, a probe where the antibody and quantum dot were coupled in the ratio of 1:1 was prepared. First, 3 μl of 0.1 mg/ml CLDN4 antibody and 0.5 μl of 1 mg/ml Protein G were mixed and then reacted at a room temperature for one hour, and followed by the addition of 7 μl of 0.2 μM quantum dots, which were then reacted at a room temperature for one hour to prepare the antibody and quantum dot in the molar ratio of 1:1.

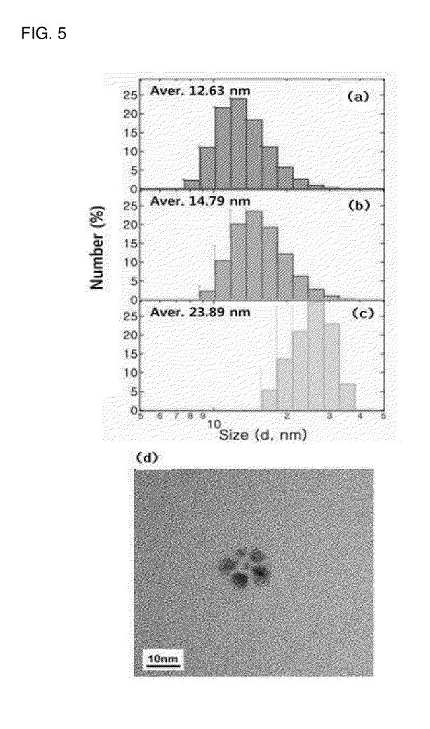

FIG. 5 is a graph showing a change in the size of the imaging probes according to the functionalization thereof, measured with DLS, in which (a) indicates the size of ferritin, (b) indicates the size of the assembly of ferritin and antibody, and (c) indicates the size of an imaging probe obtained by coupling the assembly of ferritin and antibody with a number of hydrophilic quantum dots. FIG. 5 (d) is a TEM image of the imaging probe.

### Example 2

### Preparation of Probe for Detecting Biomarker

### Streptavidin-Biotin

Biotin was added to the C-terminal of the fusion protein of Protein G and the ferritin protein prepared in Example 1-1 using PCR. The fusion protein of Example 1-1, forward primer, and backward primer were added together as shown in Table 3, and PCR conditions are as shown in Table 4.

TABLE 3

| | | SEQ ID |
|---|---|---|
| Designation | Sequence 5′→ 3′ | NO: |
| Forward Primer 6 | CATATGACGACCGCGTCCACCTCG | 6 |
| | CAGGTGCGCCAGAACTACCACCA | |
| | GGACTCAG | |
| Backward Primer 7 | CTCGAGATTAGTGATGATGCCATT | 7 |
| | CAATTTTTTGTGCCTCAAATATATC | |
| | ATTTAA | |

TABLE 4

| Segment | Number of Cycles | Temperature | Time |
|---|---|---|---|
| 1 | 30 | 95° C. | 4 min, 30 sec |
| 2 | 30 | 60° C. | 30 sec |
| 3 | 30 | 72° C. | 40 sec |
| 4 | 1 | 72° C. | 7 min |
| 5 | 1 | 4° C. | ∞ |

Next, Streptavidin was exposed at the surface of quantum dots and coupled to Biotin on ferritin. The process of exposing Streptavidin at the surface of the quantum dots is as follows. First, the surface of the quantum dots was coated with a lipid containing an SH group. 50 to 95 mole % of MHPC, 5 to 35 mole % of DPPE-PEG2000, and 1 to 15 mole % of 1,2-dipalmitoyl-sn-glycero-3-succinate containing an SH group were mixed to confer the SH group on the surface of the quantum dots. After that, the addition of SMCC rendered the SH group of the surface of the quantum dots and the maleimide group of an end portion of SMCC to be bonded by S—S disulfide bond. Lastly, the addition of Streptavidin rendered an amine group located at the other terminal of SMCC and a carboxyl group at the C-terminal of Streptavidin to be coupled, thereby exposing Streptavidin at the surface of the quantum dots.

The thus prepared fusion protein to the C-terminal of which Biotin was added and quantum dots at the surface of which streptavidin was exposed were coupled to each other by Biotin-Streptavidin reaction.

### Example 3

### Detection of Biomarker

“CLDN-4” biomarker was targeted, using the target-specific probe prepared using CLDN-4 antibody as a targeting agent. CLDN-4 has been known as a typical biomarker of pancreatic cancer.

In particular, the probe was reacted to Capan-1 pancreatic cells which were fixed with 10% formaldehyde, at a room temperature. After one hour, the surface of the cells was washed repeatedly three times with PBS solution to eliminate uncoupled probes. The thus prepared image was observed using a fluorescence microscopy.

FIG. 6 is fluorescence images observed after the biomarkers at the surface of cells/tissues were targeted using the imaging probe. (a) is a fluorescence image when an imaging probe where one fluorescent dye is coupled to one antibody is used, and (b) is a fluorescence image when an imaging probe where one quantum dot is coupled to one antibody is used. (c) is a fluorescence image when an imaging probe for super sensitivity detection where a number of quantum dots are coupled to one antibody is used. The fluorescence image obtained when the imaging probe for super sensitivity quantity analysis was used as in FIG. 6 (c) was brighter by 27.1 times than the image obtained when the fluorescence dye was used as shown in FIG. 6 (a) and it was brighter by 4.6 times than the image obtained when one quantum dot was used as shown in FIG. 6 (b).

### Example 4

### Quantity Analysis of Biomarker Detection

Fluorescence intensity values according to the concentrations of the quantum dots at concentrations of 1, 2, 3, 4, 5, 10, 15, 20, and 30 pmol/ml were measured using a fluorescence microscopy and as a result, the fluorescence intensity values were each 1335.2, 2670.4, 4005.6, 5340.8, 6676, 13352, 20028, 26704, and 40056, and a standard curve based on this was prepared as shown in FIG. 7. It was confirmed through this that a graph showing linear relationship between the concentrations of the quantum dots and the values of fluorescence intensity was obtained and accordingly, there is proportional relation between the concentrations of the quantum dots and the values of fluorescence intensity.

The amounts of biomarkers were able to be quantitatively analyzed using the standard curve. As a result of the comparison of fluorescence intensity values, the value of fluorescence intensity was 13,550 when the imaging probe for super sensitivity quantity analysis was used in FIG. 6 (c), and it was 2,945 when one quantum dot was used in FIG. 6 (b). It was confirmed through this that when the imaging probe for super sensitivity quantity analysis was used, a brighter image by 4.6 times could be obtained than when one quantum dot was used.

### Example 5

### Kit for Detecting Biomarker

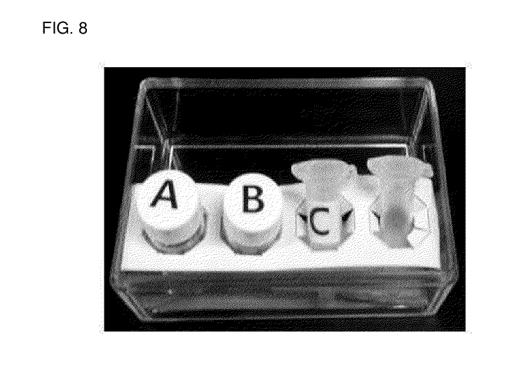

Regardless of the kinds of biomarkers, they could be quantitatively analyzed with accuracy by using a system for measuring the fluorescence intensity of quantum dots after a biomarker was targeted using ferritin and quantum dots developed in this invention, and an imaging kit was manufactured using this. FIG. 8 is an actual photograph showing an imaging kit for quantity analysis, which consists of three components. A is “a ferritin fusion protein solution where Protein G is expressed”, B is “quantum dots for quantity analysis”, and C is “a reaction vessel.”

Specific experiment methods are substantially the same as example 3 and particularly, the imaging kit was used as follows: 20 μl of ferritin solution A was added to reaction vessel C, to which 3 μl of 0.1 mg/ml CLDN4 (claudin-4) anti-human antibody solution was then added as an antibody of a biomarker to be detected and reacted at a room temperature for one hour. Next, 100 μl of quantum dot solution for quantity analysis B was added and reacted at a room temperature for one hour. After targeting the reaction solution at the surface of the desired cells/tissues, fluorescence intensity was measured and compared with the standard curve of FIG. 7 so that the biomarker was able to be quantitatively analyzed with super sensitivity. FIG. 7 is a quantum dot concentration-fluorescence intensity related standard curve obtained by analyzing various concentrations of quantum dots and resultant fluorescence intensities according to one embodiment of the invention.

What is claimed is:

1. A target-specific probe comprising a ferritin complex containing a ferritin protein and a coupling partner connected to the ferritin protein where the coupling partner does not inhibit ferritin structure formation; and a targeting agent coupled to the ferritin complex.

2. The target-specific probe of claim 1, wherein the coupling partner is Protein G, Fc receptor, Protein A, Protein A/G, Biotin, Avidin, or Streptavidin.

3. The target-specific probe of claim 1, wherein the targeting agent is an antibody, aptamer, aptide, or peptide.

4. The target-specific probe of claim 1, wherein the ferritin complex is a fusion protein containing the ferritin protein and a protein or a peptide which is connected to the C-terminal or N-terminal of the ferritin protein and does not inhibit ferritin structure formation, as the coupling partner.

5. The target-specific probe of claim 4, wherein the fusion protein contains Protein G or Fc receptor and the ferritin protein connected from its N-terminal to C-terminal, and the Fc portion of an antibody as the targeting agent is connected to Protein G or Fc receptor of the fusion protein.

6. The target-specific probe of claim 1, comprising HIS tag, CYS tag, GST tag, Biotin tag, Avidin tag, or Streptavidin tag which is connected to the terminal of the ferritin protein or the terminal of the coupling partner.

7. The target-specific probe of claim 1, further comprising a detectable labeling agent coupled to the ferritin complex.

8. The target-specific probe of claim 7, wherein the labeling agent is coupled to the ferritin complex using HIS tag, CYS tag, GST tag, Biotin tag, Avidin tag, or Streptavidin tag which is connected to the ferritin complex, as a linker.

9. The target-specific probe of claim 7, wherein the labeling agent is a quantum dot, magnetic bead nanoparticle, gold nanoparticle, fluorescent dye, fluorescent protein, nano phosphor, or silicon nanoparticle.

10. The target-specific probe of claim 7, wherein the labeling agent is coupled in 2 to 15 molar ratios with regard to 1 mole of the ferritin complex.

11. The target-specific probe of claim 9, wherein the quantum dot comprises a hydrophilic surface layer by treatment with an amphiphilic substance having a hydrophilic group and a hydrophobic group in one molecule.

12. The target-specific probe of claim 11, wherein the amphiphilic substance is one or more selected from the group consisting of MHPC, DPPE-PEG2000, Ni-NTA, and a mixture thereof.

13. The target-specific probe of claim 1, wherein comprises the ferritin complex containing the ferritin protein and a Biotin tag, Avidin tag, or Streptavidin tag compound coupled to the ferritin protein as the coupling partner; and
a compound coupled to the ferritin complex, and being capable of chemically binding to the compound coupling partner as a detectable labeling agent.

14. A method for detecting a biomarker, comprising contacting the target-specific probe according to claim 7 which contains a ferritin complex, a targeting agent specific to the biomarker, and a labeling agent, with a sample containing the biomarker; and detecting the labeling agent of the target-specific probe.

15. The method for detecting of claim 14, further comprising a step in which the biomarker is determined to be present in the sample when the labeling agent is detected.

16. The method for detecting of claim 14, wherein the method comprises the steps of:
(a) obtaining a standard curve by using the detected signal of the labeling agent measured at various concentrations of the labeling agent;
(b) detecting a signal of the labeling agent targeted to the biomarker in the sample; and
(c) comparing the signal of the labeling agent in step (b) with that in the standard curve, to obtain the biomarker amount in a sample.

17. The method for detecting of claim 14, wherein fluorescence intensity emitted by quantum dots as the labeling agent is detected in the detecting step.

18. A detection kit of a biomarker, comprising the target-specific probe according to claim 7 which contains a ferritin complex, a targeting agent specific to the biomarker, and a labeling agent; and a detector for detecting the labeling agent of the target-specific probe targeted to the biomarker.
